(19) United States
(12) Patent Application Publication
(10) Pub. No.: US 2023/0320844 A1
(43) Pub. Date: Oct. 12, 2023

(54) Apparatus and Method for Transcardiac Valve Delivery

(71) Applicant: ConneX BioMedical, Inc., Aurora, CO (US)

(72) Inventors: Christopher E. Banas, Breckenridge, CO (US); Jeffrey N. Steinmetz, Arvada, CO (US)

(21) Appl. No.: 18/189,160

(22) Filed: Mar. 23, 2023

Related U.S. Application Data
(60) Provisional application No. 63/323,058, filed on Mar. 23, 2022.

(51) Int. Cl. A61F 2/24

(52) U.S. Cl. CPC A61F 2/2412; A61F 2/2427; A61F 2220/0075

(57) ABSTRACT

An access port, access port delivery system, and a method of delivering a transcatheter mitral valve prosthesis to a mitral valve annulus within a mammalian heart. The access port includes an annular ring having a central opening, a tubular projection extending from the annular ring and coaxial with the central opening, a plurality of tissue anchor openings spaced apart about a circumference of the annular ring, and a plurality of tissue anchors. The access port delivery system removably couples to the access port and carries the plurality of tissue anchors and includes a driver mechanism for synchronously driving the plurality of tissue anchors through the plurality of tissue anchor openings to affix the access port to a ventricular apex of the heart. The access port delivery system also includes a central bore that serves as a working channel to deliver a transcatheter mitral valve prosthesis.

### CROSS-REFERENCE TO RELATED APPLICATIONS

This application is related to and claims priority to co-pending U.S. provisional patent application Serial No. 63/323,058, filed Mar. 23, 2022.

### BACKGROUND OF THE INVENTION

The present disclosure relates generally to an access port configured to be affixed to anatomic tissue and an access port delivery system. For example, any anatomic passageway that may require access to a lumen or a chamber of the anatomic passageway may be a site for affixation of the disclosed access port, e.g., lung structures, gastrointestinal structures, central nervous system structures, including central and peripheral nervous systems, or cardiovascular structures. As a particular example of the access port of the present disclosure, reference is made in the present application to a transcardiac access port affixed and retained post-procedure on the cardiac muscle, such as the ventricular myocardium, and an access port delivery system configured to deliver the access port, secure the access port to the muscle tissue with tissue anchors, and provide a working channel through the access port to facilitate transcardiac access and device delivery. In particular, the present device is well suited for delivery to the ventricular apex of the heart muscle to facilitate transapical access and device delivery.

The access port itself, consists generally of an annular ring, an optional tubular projection extending from a center axis of the annular ring, and a central opening passing through the tubular projection. The central opening defines a working opening for access to the heart muscle. Optionally, a hemostatic valve may be provided and associated with the central opening of the access port to provide hemostasis and allow passage of delivery tools, catheters, and/or other medical devices there through. The annular ring may have a substantially planar configuration or maybe a curvilinear surface. A plurality of tissue anchor openings pass through the annular ring of the access port and are positioned in a spaced apart relationship about the annular ring. A plurality of tissue anchors are configured to pass into and through the plurality of tissue anchor openings and apply an axially compressive force to the access port against the epicardium and myocardium of the heart.

Alternatively, the access port may be configured in a manner similar to the apical cuff for the axial compression apical cuff assembly described in U.S. Patent No. 11,123,542 for attachment of ventricular assist device pump (the “‘542 Patent”), which is hereby incorporated in its entirety. In this alternative embodiment, the annular ring of the access port may have at least one of a plurality of recesses or openings, and an axial compression ring that has the plurality of tissue anchor openings is configured to fit within the at least one of a plurality of recesses or openings in the annular ring, with the plurality of tissue anchors then passing into and through the tissue anchor openings and exerting an axially compressive force to the axial compression ring and to the access port against the epicardium and myocardium.

The delivery system consists generally of synchronous drive system that includes a drive input sub-system, a drive gear sub-system, a targeting sub-system aligned along a central longitudinal axis of the delivery system that penetrates the epicardium and is configured to provide targeting and alignment of the access port to a desired position on the heart, and a driver sub-system that retains the access port and tissue anchors during delivery of the access port, and synchronously drives the tissue anchors into and through the tissue anchor openings in the access port and into the heart, and. All of the foregoing sub-systems are oriented about a central tubular bore that supports the foregoing sub-systems and acts as a working channel that allows passage of the TMVR delivery system. A handle housing is provided that is coupled to the foregoing sub-systems and facilitate the surgeon to manipulate the delivery system. The drive input sub-system extends to a proximal end of the handle housing, and has a rotational input member, such as a rotational handle, knob, or stem, with the tubular central bore being accessible from a proximal end of the rotational input member to allow access to the working channel from the input member. Optionally, the delivery system includes the access needle, hemostatic valve, dilator, and a guidewire lumen associated with the central bore.

One example of transcatheter transapical medical devices are cardiac valves, such as the TENDYNE (Abbott Laboratories, Abbott Park, Illinois) transcatheter mitral valve replacement (TMVR). Transcatheter mitral valve replacement entails either intraluminal or transcardiac access to the intracardiac structural apparatus, then delivery and placement of the mitral valve prosthesis. Some TMVR devices engage and anchor to the mitral annulus, e.g., CARDIAQ (Edwards Lifesciences Corporation, Irvine, California), the fibrous trigonum of the heart, e.g., TIARA (Neovasc, Inc., Richmond, British Columbia, Canada), or by radial force and subannular cleats, e.g., INTREPID (Medtronic, Minneapolis, Minnesota). The Abbott TENDYNE TMVR device and delivery is described in U.S. Patents Nos. 9,827,092; 10,327,894; 10,405,976; 10,610,356; 10,786,351; and/or 11,090,157, each of which is hereby incorporated by reference. Delivery methods and delivery devices for delivery and deployment of the TENDYNE TMVR are described in U.S. Patents Nos. 10,517,728; 11,039,921; and/or 11,246,562, each of which is herein incorporated by reference.

A further example of a transapical medical device for which the access port of the present disclosure may be used is in placing a ventricular assist device. Examples of transapical VAD and LVAD devices which may be employed with the access port of the present disclosure include, without limitation, U.S. Patent Nos. 9,138,517; 9,919,089; 10,485,910; 10,933,180, 11,097,093; 11,376,414; 11,452,861,11,197,618, 10,933,318, 10,11,097,091, 9,186,447, 8,807,968, 8,409,276, or 7,993,260.

For purposes of illustration only, and without intention to limit the use or application of the present invention to TMVR procedures, the present disclosure references the TMVR procedure and the TENDYNE TMVR specifically. It will be understood, however, that a wide variety of other applications and uses of the access port of the present invention, including, for example, other mitral valve repair or replacement procedures, aortic valve repair or replacement, such as a transcatheter aortic valve replacement (TAVR) procedure, septal defect repair, valve or valve structure repair, ventricular assist device placement, or any other procedure in which access to the intracardiac anatomy may be required. An advantage of the disclosed access port lies in its utility for multiple successive accesses to the intracardiac anatomy during the course of cardiac function diminution. For example, placement of the disclosed access port for a TMVR procedure may be followed sometime later by a need for access for aortic valve replacement or ventricular assist device placement.

In accordance with the TENDYNE TMVR instructions for use, the delivery and deployment process for the TENDYNE TMVR is generally as follows: a mini-thoracotomy is performed on the patient to access the ventricular apex of the heart; apical access is established using transesophageal echocardiography and the Seldinger technique using a 0.035″ (0.889 mm/ 2.67 French) guidewire; pledgeted sutures are placed around the apical access site, the access site is then dilated and a balloon catheter is introduced into the left ventricle and advanced over the guidewire into the left atrium; the balloon catheter is removed, leaving the guidewire positioned in the left atrium, the delivery system carrying the TENDYNE TMVR which is advanced over the guidewire, and positioned superior to the mitral valve annulus; the delivery system dilator is collapsed and removed with the guidewire, and the TMVR is deployed in the mitral valve annulus, once proper positioning is confirmed, the delivery system is withdrawn releasing and maintaining tension on the tether coupled to the TMVR until the tether is exposed outside the epicardium, then an apical pad and positioning handle is threaded onto a distal end of the tether, and advanced, while maintaining tension on the tether to maintain valve position in the mitral valve annulus, under the apical pad and handle contact the epicardium; valve position and performance are then confirmed and the tether tension adjusted as necessary, then the apical pad positioned is then fastened with an epicardial anchoring device, to secure the tension on the tether and the proper positioning of the valve in the mitral valve annulus. The foregoing delivery process is described in greater detail in U.S. Patent No. 10,517,728, incorporated by reference.

In accordance with one aspect of the present disclosure, when the access port and delivery system of the present disclosure is employed, the access port is first attached to the epicardium and myocardium of the ventricular apex. Once placed, the delivery and deployment of the TENDYNE TMVR may be achieved through a central opening of the access port. In accordance with the present invention, the access port is similar in concept to the axial compression apical cuff assembly described in U.S. Patent No. 11,123,542 for attachment of ventricular assist device pump (the “‘542 Patent”), which is hereby incorporated in its entirety.

In accordance with one variant of the access port of the present disclosure the access port includes an annular ring, an optional tubular projection extending co-axially with the annular ring, a hemostasis ring underlying the annular ring, a central opening that passes through the optional tubular projection or through the annular ring, when the tubular projection is not employed, and a plurality of tissue anchors. In this manner the central opening provides a working channel through which an intracardiac procedure may be performed. The plurality of tissue anchor openings pass through the annular ring of the access port and are positioned in a spaced apart relationship about the annular ring. The plurality of tissue anchors are configured to pass into and through the plurality of tissue anchor openings, into and through the hemostasis ring, and apply an axially compressive force to the access port against the epicardium and myocardium of the heart.

When provided, the tubular projection has an engagement configured to removably couple to an access port delivery system. The tubular projection may also extend distally relative to the annular ring and have a length such that the tubular projection does not extend beyond the endocardium, or the innermost layer that lines the inner wall surface of the ventricle.

In accordance with an alternative variant of the access port, the access port may be configured in a manner similar to the apical cuff of the axial compression apical cuff assembly described in the ‘542 Patent. In this alternative embodiment, the annular ring of the access port may have at least one of a plurality of recesses or openings, and an axial compression ring have the plurality of tissue anchor openings is configured to fit within the at least one of a plurality of recesses or openings in the annular ring, with the plurality of tissue anchors then passing into and through the tissue anchor openings and exerting an axially compressive force to the axial compression ring and to the access port against the epicardium and myocardium.

A delivery system, such as that described in U.S. Patent Application Serial No. 17/351,082, filed Jun. 17, 2021, (the “‘082 Patent”) and incorporated by reference in its entirety, is useful to deliver and affix the disclosed access port to the heart, has a central lumen that may be used as a guidewire channel and/or a working channel, and allow for closure of the central opening of the access port.

The delivery system consists generally of synchronous drive system that includes a drive input sub-system, a drive gear sub-system, a driver sub-system that retains the access port and tissue anchors during delivery of the access port, and synchronously drives the tissue anchors into and through the tissue anchor openings in the access port and into the heart, and a targeting sub-system aligned along a central longitudinal axis of the delivery system that penetrates the epicardium and is configured to provide targeting and alignment of the access port to a desired position on the heart. All of the foregoing sub-systems are oriented about a central tubular bore that supports the foregoing sub-systems and may be configured to serve as a working channel that allows passage of the TMVR or other device delivery system. A handle housing is provided that is coupled to the foregoing sub-systems and facilitate the surgeon to manipulate the delivery system. The drive input sub-system extends to a proximal end of the handle housing, and has a rotational input member, such as a rotational handle, knob, or stem, with the tubular central bore being accessible from a proximal end of the rotational input member to allow access to the working channel from the input member.

### SUMMARY OF THE INVENTION

It is an object of the present disclosure to provide an access port that is removably coupled to an access port delivery system, to both allow for delivery of the access port, synchronous driving of tissue anchors into the heart to rapidly affix the access port to the heart.

It is a further object of the present disclosure to configure the access port delivery system to provide a working channel that permits a device delivery system to pass through the access port delivery system and allow placement of the device in an intracardiac space or cardiac valve annulus and sealing of the access port.

It is another object of the present disclosure to provide an access port having an annular ring, an optional tubular projection extending co-axially with and from the annular ring, and a central opening passing through the optional tubular projection and/or the annular ring defining a working opening for access to the heart muscle.

Another object of the present invention that the optional tubular projection be removably engageable with the annular ring.

It is another further object of the present disclosure to provide a hemostatic seal in the central opening of the access port and/or in the optional tubular projection.

It is a further object of the present disclosure to configure the optional tubular projection to be removably engageable with a delivery system to provide engagement between the access port and the delivery system during the procedure.

It is still another object of the present disclosure to provide a plurality of tissue anchor openings pass through the annular ring of the access port and are positioned in a spaced apart relationship about the annular ring.

It is yet another object of the present disclosure to provide a plurality of tissue anchors configured to pass into and through the plurality of tissue anchor openings and apply an axially compressive force to the access port against the epicardium and myocardium of the heart.

It is still a further object of the present disclosure that the plurality of tissue anchors are tissue screws having an open coil configuration, are expandable pins, expandable connectors, retainers, or the like.

It is yet another further object of the present disclosure that the annular ring of the access port has a plurality of recesses or openings, and an axial compression ring having the plurality of tissue anchor openings is configured to fit within the at least one of a plurality of recesses or openings in the annular ring, with the plurality of tissue anchors then passing into and through the tissue anchor openings and exerting an axially compressive force to the axial compression ring and to the access port against the epicardium and myocardium.

It is still another further object of the present disclosure that the axial compression ring be a unitary ring structure.

It is yet still another further object of the present disclosure that the axial compression ring be a composed of segmented, separate, or interconnected arcuate plate members; where segmented or interconnected, the arcuate plate members may have an interface connection, such as a tongue and groove, a strain relief connector, or an expandable or elongating connector, thereby allowing the arcuate plate members to have a range of movement relative to one another while still providing axial compression to the access port against the heart muscle.

It is another further object of the present disclosure to provide an access port delivery system configured to deliver and affix the access port to the heart, and allow for closure of the central opening of the access port. Optionally the access port delivery system may be configured to serve as a working channel for delivery of the TMVR device, and allow for closure of the central opening of the access port.

It is still another further object of the present disclosure to provide the access port delivery system is configured to synchronously drive the plurality of tissue anchors.

It is another object of the present disclosure that the access port delivery system includes a drive input sub-system, a drive gear sub-system, a driver sub-system that retains the access port and tissue anchors during delivery of the access port, and synchronously drives the tissue anchors into and through the tissue anchor openings in the access port and into the heart, a targeting sub-system aligned along a central longitudinal axis of the delivery system that penetrates the epicardium and is configured to provide targeting and alignment of the access port to a desired position on the heart, and includes an integral needle, dilator or coring tool, and a hemostatic valve.

It is yet a further object of the present disclosure that the access port delivery sub-systems are oriented about a central tubular bore that acts as a working channel that allows passage of a TMVR or other device delivery system.

It is still a further object of the present disclosure to provide a handle housing coupled to the sub-systems configured to allow the surgeon to manipulate the access port delivery system.

It is yet another object of the present disclosure to provide a handle coupled to the drive input sub-system that has access to the working channel to allow passage of the TMVR or other device delivery system into and through the working channel from the handle.

These and other objects, features and advantages of the present invention will be more apparent to those skilled in the art from the following more detailed description of the invention with reference to the accompanying Figures.

### BRIEF DESCRIPTION OF THE DRAWINGS

FIG. 1 is a perspective view of an access port in accordance with the present disclosure.

FIG. 2 is a perspective partially exploded view of the access port with engaged tissue anchors and an access cap in accordance with the present disclosure.

FIG. 3 is a perspective view of an assembly including the access port engaged with the access cap in accordance with the present disclosure.

FIG. 4A is a perspective cross-sectional view of the assembly including the access port and its engagement with the access cap and showing an attachment skirt associated with the access port in accordance with the present disclosure.

FIG. 4B is an exploded cross-sectional view of the assembly including the access port, the attachment skirt, and the access cap in accordance with the present disclosure.

FIG. 5A is a perspective cross-sectional view of an access port illustrating engagement with an optional tubular projection.

FIG. 5B is a perspective cross-sectional view of an access port illustrating engagement with a sealing plug.

FIG. 5C is a perspective cross-sectional view of an access port illustrating engagement with an alternative sealing plug.

FIG. 5D is a perspective cross-sectional view of an access port with a one-way valve disposed in the central opening of the access port.

FIG. 5E is a perspective cross-sectional view of an access port with a one-way valve and a sealing plug disposed within the one-way valve in accordance with the present disclosure.

FIG. 6A is a diagrammatic illustration showing transapical access of a transcatheter aortic valve replacement catheter passing through the access port of the present disclosure.

FIG. 6B is a diagrammatic illustration showing transapical placement of a ventricular assist device through the access port of the present disclosure.

FIG. 7 is a diagrammatic illustration showing placement of a guidewire into the left ventricle of a heart.

FIG. 8 is a diagrammatic illustration showing engagement of an access cuff and access cuff delivery system over the guidewire placed into the left ventricle of a heart and deployment of the access port onto the ventricular apex of the heart.

FIG. 9 is a diagrammatic illustration showing engagement of a TENDYNE TMVR apical pad and apical anchor over a tether bridle passing through the central opening of the access port of the present disclosure.

FIG. 10 is a diagrammatic illustration showing engagement of the TENDYNE TMVR apical pad and apical anchor with the access cuff of the present disclosure.

FIG. 11 is a diagrammatic illustration showing the locking engagement of the TENDYNE TMVR apical pad and apical anchor with the access port of the present disclosure, with a tether bridle trimmed to desired length.

FIG. 12 is a flow diagram illustrating the method steps of delivering and affixation of the access port to the heart, and delivery of the exemplary TENDYNE TMVR through the access port of the present disclosure.

### DETAILED DESCRIPTION OF THE PREFERRED EMBODIMENTS

The device, system and methods of the present invention will be described with reference to certain exemplary embodiments thereof. These exemplary embodiments or variants are intended to be illustrative and non-limiting examples of the present invention. The example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments or variants may be embodied in many different forms and that neither should be construed to limit the scope of the disclosure. Those of ordinary skill in the art will understand and appreciate that variations in materials, structure, material properties, and tolerances may be made without departing from the scope of the invention, which is defined only by the claims appended hereto and their range of equivalents. In some example embodiments, well-known processes, well-known device structures, and well-known technologies are not described in detail.

Benefits, other advantages, and solutions to problems have been described herein with regard to specific embodiments or variants. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims where A, B, and C refer to claimed elements, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching when used throughout the figures to denote different parts but not necessarily to denote the same or different materials.

For ease of understanding, the present invention is described with reference to the accompanying Figures. In the accompanying Figures like elements are identified by like reference numerals.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature’s relationship to another element(s) or feature(s) as illustrated in the figures. Spatially relative terms may be intended to encompass different orientations of the device in use or operation in addition to the orientation depicted in the figures For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than and including totally.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision with some approach to exactness in the value; approximately or reasonably close to the value; nearly. If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

“Transabdominal” is intended to mean passing across the abdominal wall and/or abdominal cavity.

“Transcardiac” is intended to mean passing across the cardiac wall.

“Transthoracic” is intended to mean passing across the chest and/or the thoracic cavity.

Medical or anatomical terms are intended to have their usual and customary meaning in the medial arts and terminology.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.”

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

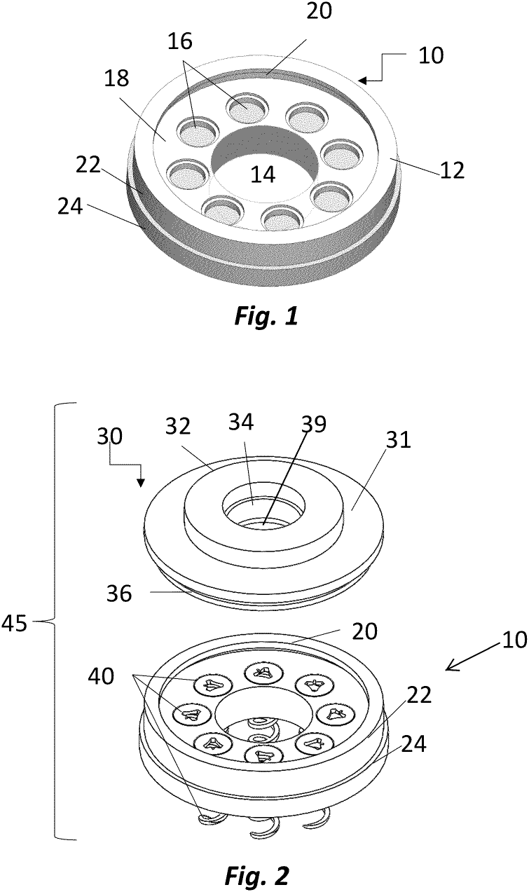

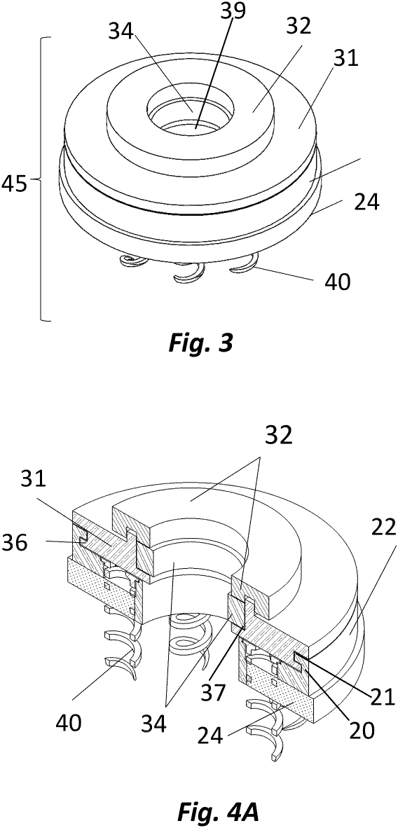

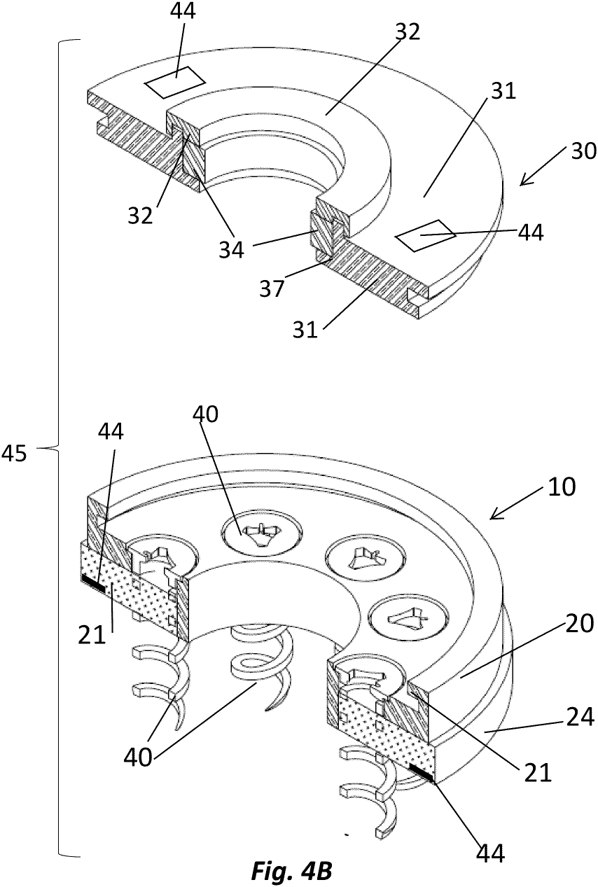

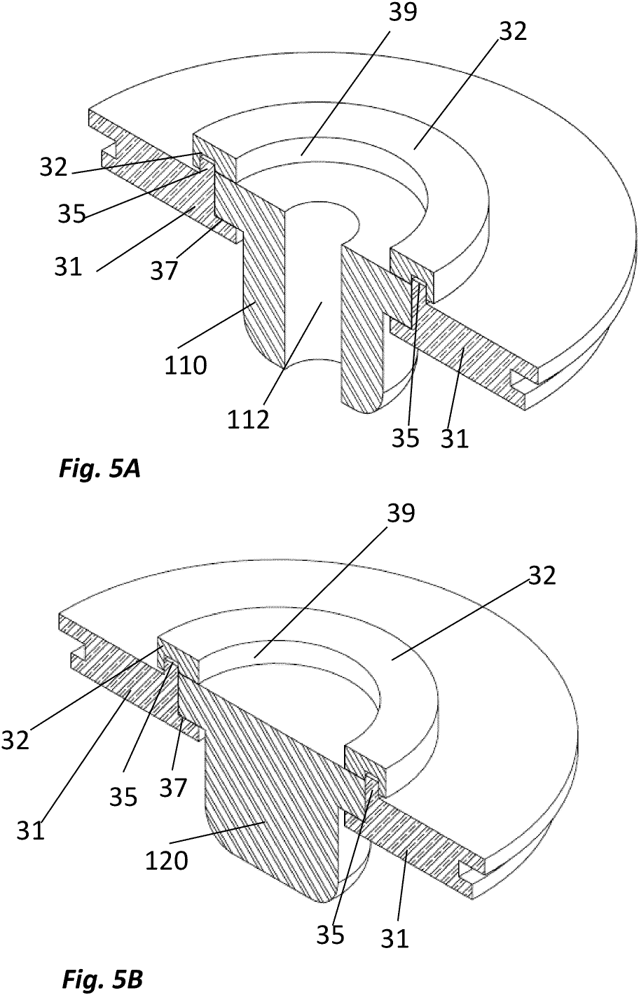

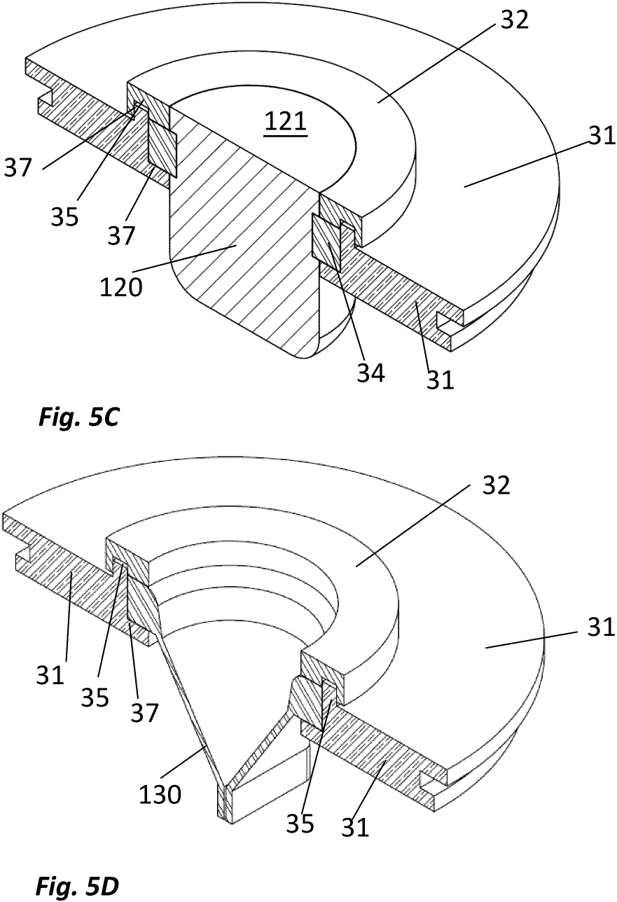

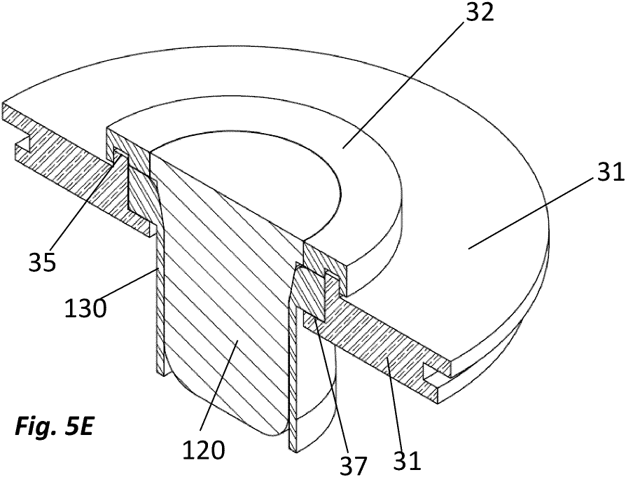

Turning to the variants of the access port of the present disclosure as illustrated in and with reference to the accompanying figures, there is described an access port assembly 45 of the present disclosure. As shown in FIGS. 1-4B, the access port assembly 45 consists generally of several component devices, namely, an access cuff 10 and an access cap 30. An attachment skirt 24 is coupled to a distal or heart-facing surface of the access cuff 10 and the access cap 30 is removably coupled to a proximal surface of the access cuff. Additionally, the access port assembly 45 may include a tubular projection 110 (See, FIG. 5A) that may serve as a sealing working channel or collar for a medical device which may be passed through the lumen of the tubular projection. The tubular projection 110 extends distally through the access port assembly 45 and into the penetration into the heart muscle tissue. Further, the assembly 45 may, optionally, include a hemostatic plug 120 (See, FIGS. 5B, 5C, and 5E), which engages with the access cap 30 and passes into and through the central opening 14 of the access cuff 10. Moreover, a one-way valve 130 (See, FIGS. 5D and 5E) may also, optionally, be provided that is retained on access cap 30 and passes into and through the central opening 14 of the access port. Finally, combinations of the tubular projection 110, one-way valve 130, and plug 120 are also intended and contemplated by the present disclosure, as exemplified in FIG. 5E showing a plug 120 coupled within the one-way valve 130 to hemostatically occlude the central opening 14 of the access cuff 10. Each of the tubular projection 110, one-way valve 130, and plug 120 are coupled to the access cap 30 and project distally from the access cap 30 and into the central opening 14 of the access cuff 10.

The tubular projection 110, one-way valve 130, and plug 120 are positioned within a recess in a central opening 39 of a first annular cap member 31 and are secured by a second annular cap member 32. The second annular cap member 32 may be joined to one or more of the tubular projection 110, one-way valve 130, seal 34, and/or plug 120 in a manner that permits simultaneous removal and insertion of the second annular cap member 32 along with one or more of the tubular projection 110, one-way valve 130, seal 34, and/or plug 120. Alternatively, one or more of the tubular projection 110, one-way valve 130, plug 120, or seal 34 may be separate elements from the second annular cap member 32 and independently removable from the first annular cap member 31.

The access cuff serves as a trans-cardiac access site for the dual lumen perfusion cannula. The access cuff 10 is configured for placement on an epicardial surface of a heart 5, such as the left or right ventricle, and allow access through the cardiac muscle tissue and into the ventricular space to allow passage of the medical device, e.g., a dual lumen perfusion cannula, through the access cuff, into the cardiac ventricle and, in the case of the perfusion cannula, extend past the associated arterial valve into the distal associated artery.

The access cuff 10 as shown in FIGS. 1, 2, 3 and 4 is configured for placement onto the epicardial surface through a minimal access incision, such as a subxyphoid epigastric incision or limited thoracotomy. The access cuff 10 is comprised of an annular port member 12, having a central bore opening 14, a plurality of tissue anchor apertures 16 passing axially through a flange 18 concentrically surrounding the central bore opening 14, and an annular attachment skirt 24 having a central opening axially aligned with the central bore opening 14 of the annular port member 12.

The annular port member 12 has an outer circumferential edge 22 having a circumferential aspect 20 projecting axially therefrom and defining an outward aspect of flange 18. The circumferential aspect 20 is configured to have a mating attachment 21 that couples to a corresponding mating surface 36 of the access cap 30. The mating attachment 21 and mating surface 36 may take a wide variety of configurations, including, without limitation, friction fit, interference fit, threaded fit, interlocking fit, bayonet fit, press-fit, or the like.

The attachment skirt 24 has a first surface that is positioned to abut flange 18 on a heart-facing surface thereof. A second surface of attachment skirt is positioned to abut the cardia tissue 5. The attachment skirt 24 facilitates hemostasis with surgical attachment to a cardiac surface 5 and may be comprised of a flexible or rigid material. Examples of suitable materials for the attachment skirt 24 are polytetrafluoroethylene (PTFE), polyethylene terephthalate (PET), or similar non-woven or woven material suitable for creating aiding hemostasis and axial compression of the cuff assembly to the heart muscle. The attachment skirt 24 has a plurality of tissue anchor receiving sites arrayed about a circumferential aspect of the attachment skirt and is configured to allow for tissue anchors to penetrate into and through the attachment skirt 24 without substantial bleeding.

Tissue anchors 40 engage perpendicularly into and through the tissue anchor receiving sites on the attachment skirt 24 and are deployed either individually, in groups, or simultaneously to couple the access cuff 10 to the ventricular surface. The tissue anchors 40 and attachment skirt 24 exert an axially compressive force to secure the access cuff 10 to the epicardial surface 5 of the ventricle with hemostasis.

While the tissue anchors 40 shown in the accompanying Figures are helical coil tissue screws, it will be understood that alternative tissue anchors, including, for example, sutures, barbs, pins, or the like, are also useful as tissue anchors 40 in the present disclosure.

The access cuff 10, tissue anchors 40, and attachment skirt 24 are conceptually similar to the apical cuff attachment system disclosed in the ‘542 Patent, supra, and U.S. Published Patent Application Publication No. US 2022-0249831, which are both hereby incorporated by reference in their entirety.

The access cap 30 and its variants shown FIGS. 2-5E is either fixedly or detachably coupled to the access cuff 10. The access cap 30 has a first annular cap member 31 has an engagement 21 that is configured to mate with a circumferential aspect 20 of the access cuff 10. To facilitate a hemostatic engagement there between, first annular cap member 31 may have a mating surface 36 which cooperates with mating attachment 21 of the access cuff 10, ideally in a hemostatic manner. A second annular cap member 32 engages with the first annular cap member 31 and serves to retain a seal 34 centrally positioned within the central annular opening formed by axial alignment of first annular cap member 31 and the second annular member 21. Access cap 30 has a central opening 39 configured to allow a medical device to pass through the access cap 30 by passing through the second annular cap member 32, the seal 34, which seals against the medical device, then passing through the first annular cap member 31 and into and through the access cuff 10 and into the heart 5. Seal 34 is centrally positioned in the access cap 30 and is configured to bear against an outer wall surface of a medical device passed through the central opening 39, and both allow the medical device to be axially adjusted through the seal 34 and provide a hemostatic seal around the outer wall surface of the medical device once it is in a desired position. Seal 34 may be a circumferential seal such as a Tuohy-Borst valve or an O-ring seal or may be a one-way valve such as dome valve, a duckbill, or multi-cuspid valve seal. Seal 34 is seated against or joined to the second annular cap member 32 and may be configured to be removable with the second annular cap member 32 from the first annular cap member 31.

Illustrated in FIGS. 5A-5E are variants of the access cap 30 in which an additional element is coupled to the secondary annular member 32, and the second annular cap member 32 removably couples to the first annular member. According to a first variant, shown in FIG. 5A, a tubular projection 110 is coupled to the second annular cap member 32 and extends into and through the central bore opening 14 in the annular port member 12. The tubular projection 110 has at least one lumen 112 that extends the length of the tubular projection 110 and is co-axial with the central opening 39 of the cap member 30 and with the central bore opening of the annular port member 12. The tubular projection 110 may have a length that extends through the ventricular wall and, optionally, into the intraventricular chamber.

According to a second variant, shown in FIG. 5B, a sealing plug 120 is coupled to the second annular cap member 32 and extends co-axially through both the central opening 39 of the cap member 30 and the central bore opening 14 of the annular port member 12. The sealing plug 120 completely occludes both the central opening 39 of the second annular cap member 32 and the central bore opening 14 of the first annular cap member 31. The sealing plug 120 optionally has a length that extends into the ventricular wall but does not extend into the intraventricular space such that it at least substantially occludes the penetration into the ventricular wall thickness. In this manner, little to now space is left in which potentially dangerous thrombus may form. Sealing plug 120 may optionally be made of a thrombogenic or a tissue growth scaffold material to promote occlusion of the tissue penetration and healing of the sealing plug 120.

According to a third variant, shown in FIG. 5C, the sealing plug 120 is coupled to the second annular cap member 32 and retained by seal 34 within the central opening 39 of the second annular cap member. In this variant, the sealing plug 120 has an upper surface 121 that is at least substantially co-planar with an upper surface of the second annular cap member 32. This combination of sealing plug 120 and seal 34 further aids in creating a hemostatic seal at the ventricular penetration site, while allowing for subsequent removal of the sealing plug 120 for subsequent cardiac access procedures.

According to fourth variant, shown in FIG. 5D, a valve 130 is disposed in the central opening 39 of the second annular member 31. The hemostatic valve 130 is preferably a one-way valve, such as a Touhy-Borst valve, poppet valve, or a disk-valve, which prevents bleeding through the valve from the ventricle. Valve 130 seats against recess 37 in the first annular cap member 31, extends into and through the central opening 39 of the first annular cap member 31, and is secured by the second annular cap member 32. In this manner, valve 130 extends distally from the first annular cap member 31 and into the central opening 14 of the access cuff 10.

According to a fifth variant, shown in FIG. 5E, sealing plug 120 is coupled within the valve 130 to further seal the central opening 14 of the access cuff 10. As discussed above, in this fifth variant, both the valve 130 and the sealing plug 120 optionally have lengths that extend into the ventricular wall but do not extend into the intraventricular space such that it at least substantially occludes the penetration into the ventricular wall thickness. In this manner, little to now space is left in which potentially dangerous thrombus may form.

Finally it will be understood that combinations of the foregoing variants may be employed. For example, the hemostatic valve 130 may be positioned in the tubular projection 110 and the procedure accomplished through both the tubular projection 110 and the hemostatic valve 130. Alternatively, the sealing plug 120 may be placed through the hemostatic valve 130, in order to maintain hemostasis while placing the sealing plug 120. Those skilled in the art will understand that other combinations of the foregoing variants are contemplated and intended as well.

Following patient recovery or transplant, the access port 45 is configured accept and secure the hemostatic sealing plug 120 that can be inserted via repeat minimally invasive exposure and coupled to the access port 45 to provide a hemostatic seal of the central bore opening 14 of the access port 45. The access port 45 is capable of being produced in a wide variety of sizes and central opening diameters to accommodate a range of cannulas, catheters, instruments, devices, and patient sizes.

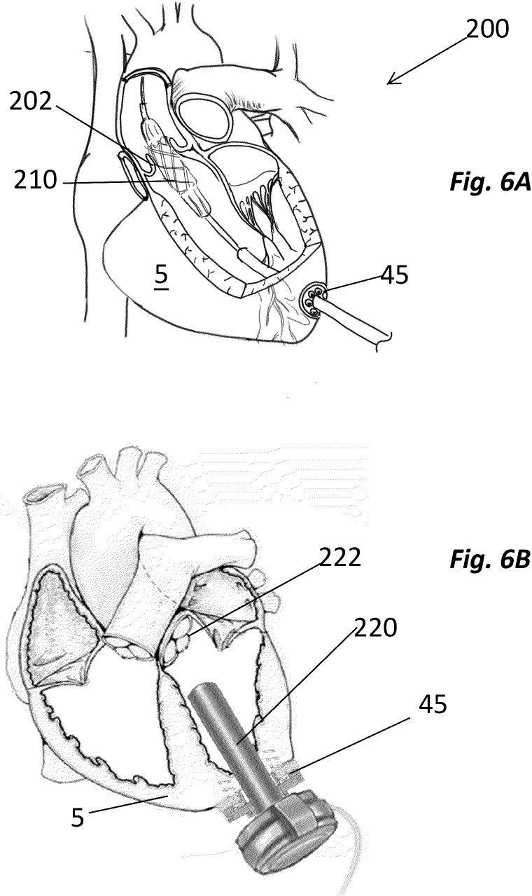

FIG. 6A depicts transapical delivery of a TAVR through the access port 45. During the TAVR delivery procedure, the TAVR delivery catheter passes through a hemostatic valve (not shown) in the central bore opening 14 of the access port 45. This configuration ensures hemostasis during the TAVR delivery procedure. It will be understood further that once the TAVR is delivered to the aortic valve annulus 202 and the TAVR delivery system removed from the intracardiac space through the access port 45, the hemostatic valve will maintain hemostasis until a hemostatic plug is joined to the access port to seal the central bore opening 14 and the cardiac tissue at the apical access site.

FIG. 6B depicts transapical delivery of a VAD 220, such as the Fineheart, SA ICOMS FLOWMAKER (Fineheart, S.A., Pessac, France), through the access port 45. During delivery of the VAD 220, the VAD 220 is placed through the central bore opening 14 of the access port 45 and positioned within the intraventricular chamber of the heart 5 to orient outflow from the VAD 220 toward the aortic valve 202.

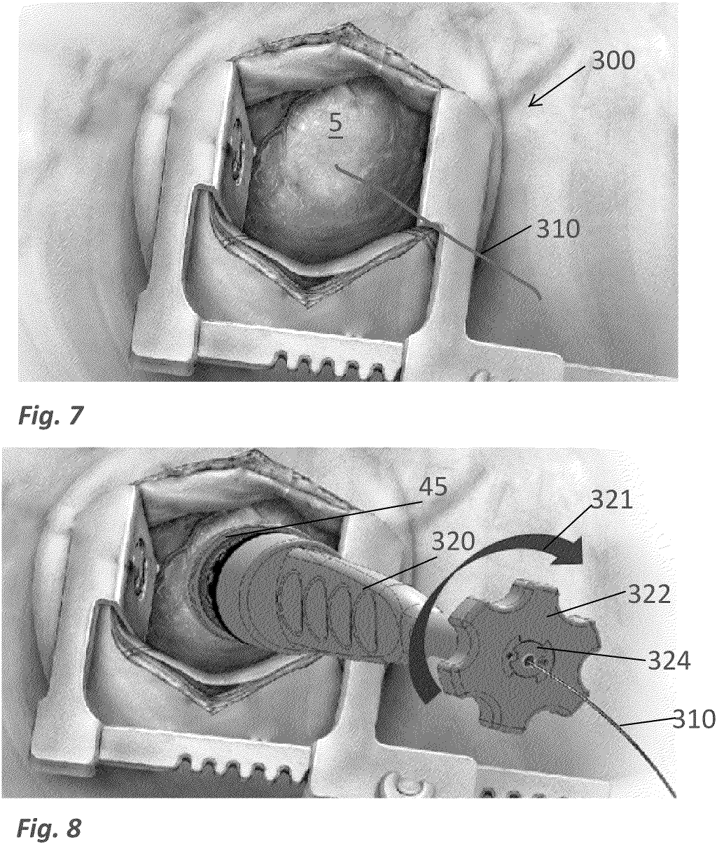

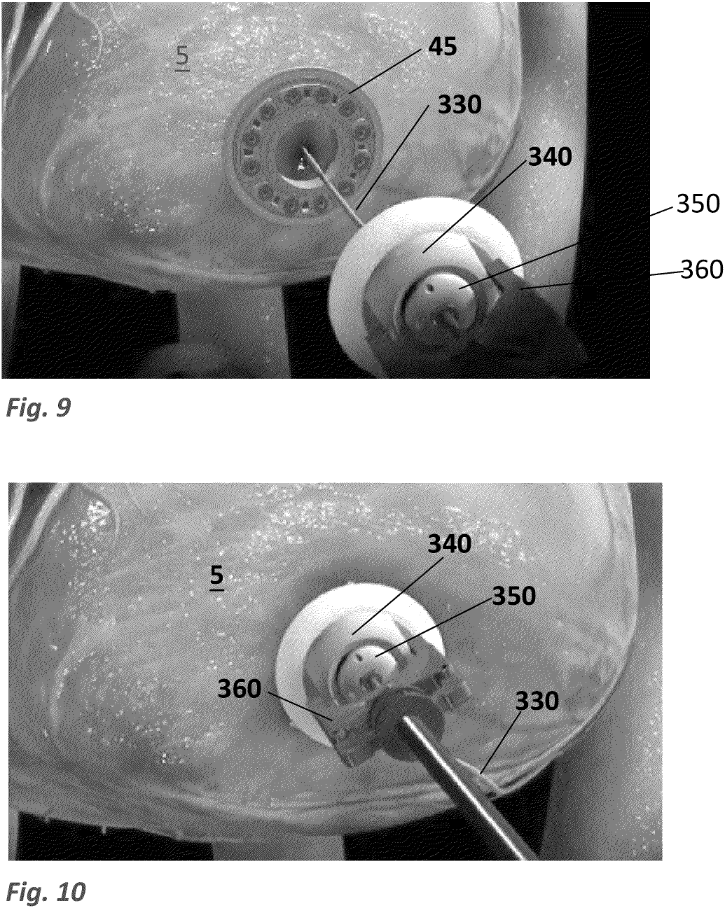

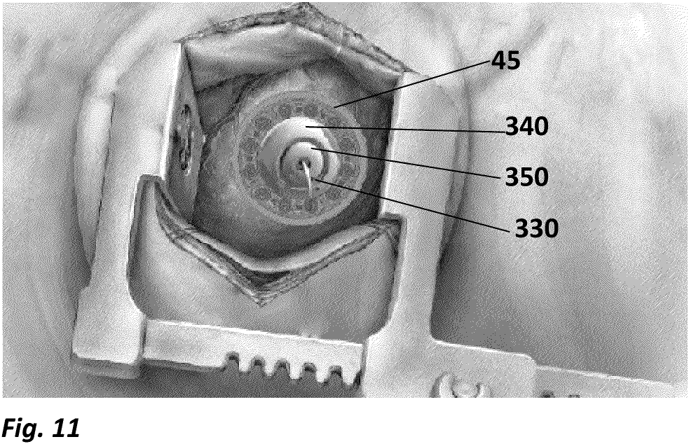

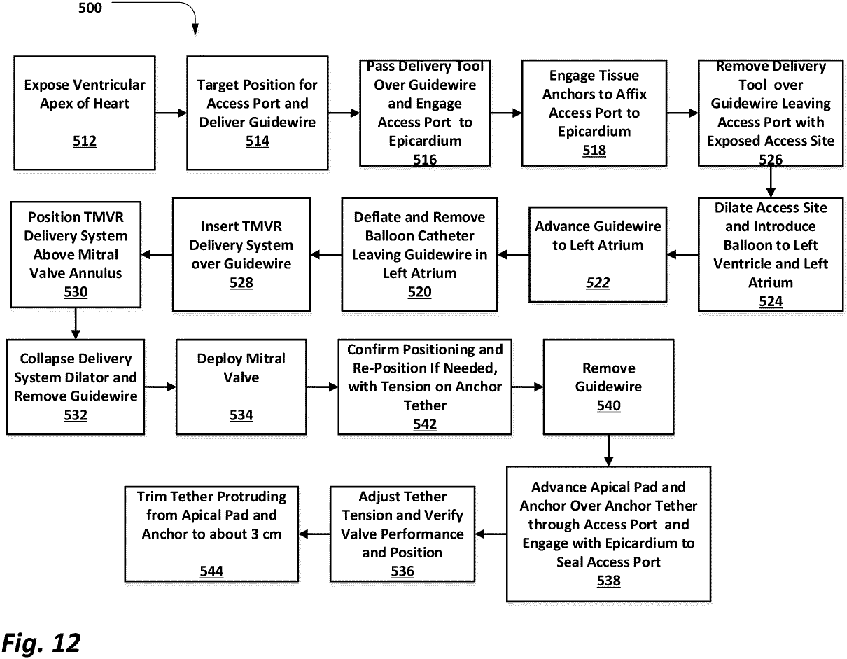

FIGS. 7-11 illustrate a method and use of the inventive apical access port with the TENDYNE TMVR and FIG. 12 is a flow diagram setting forth the method steps for delivering the TENDYNE TMVR through the disclosed access port.

The TENDYNE TMVR has a mitral valve component, a tether coupled to an inferior aspect of the mitral valve component, and an anchor pad and anchor at a distal end of the tether. When placed, the tether of the TENDYNE TMVR extends through the ventricular apex myocardium and through the epicardium, and the anchor pad and anchor are coupled to the tether and maintain tension on the tether to maintain the position of the mitral valve component in the mitral valve annulus.

As shown in succession in FIGS. 7-11, the ventricular apex of the heart is exposed via a mini-thoracotomy, which may be performed sub-xyphoid. Once the access site is exposed, a guidewire is placed through the pericardium and into the intraventricular space, as shown in FIG. 7. The access port delivery system with the access port removably attached is then passed over the guidewire. As shown in FIG. 8, the guidewire passes through a central bore in the access port delivery system thereby self-centering the access port on the guidewire and the access site. Once the access port is brought into contact with the pericardium and the access port delivery system is actuated to synchronously drive the tissue anchors into and through the access port and into the heart tissue, thereby securing the access port to the heart.

The access port delivery system may then be removed, and the TENDYNE TMVR procedure is performed through the central opening of the access port in accordance with the TENDYNE TMVR instructions for use, i.e., a balloon catheter is placed over the guidewire and passed through the mitral valve annulus, then the guidewire is advanced past the balloon and into the left atrium superior to the mitral valve. Once the guidewire is positioned in the left atrium, the balloon is deflated, and the balloon catheter removed. The TENDYNE TMVR delivery system is then passed over the guidewire and into the right atrium, the guidewire is removed, and the TENDYNE TMVR is deployed within the mitral valve annulus. The TENDYNE TMVR tethers are retained within the TENDYNE TMVR delivery system and tension is maintained on the tethers by way of a tether bridle 330 as the delivery system is withdrawn. The TENDYNE apical pad is then placed over the tether bridle 330 and engaged within and locked either onto a mating connector on the access port 45, to a mating connector on the optional tubular projection, or to a mating connector associated with the central opening 14 of the access cuff 10, and /or the central opening 39 of the access cap 30. The tether bridle 330 is passed through a locking nut that engages with the TENDYNE apical pad to secure the tether bridle 330 under a desired tension, then the tether bridle 330 is trimmed to a desired length.

FIG. 12 is a method flow diagram illustrating the method 500 for delivery of the access port of the present invention. The heart, particularly, the ventricular apex of the heart is exposed 512 such as by a mini thoracotomy, as shown in FIG. 7. Once the heart is exposed, a guidewire is placed through the heart muscle and into the ventricular space 514. The access port delivery system is passed over the guidewire placed at the ventricular apex 516 and the access port delivery system is actuated to affix the access port to the surface of the heart as shown in FIG. 8 by engaging the tissue anchors 518. The access port delivery system is then removed over the guidewire leaving the access port affixed to the heart with the access site and guidewire exposed through the central opening of the access port 520. The TENDYNE TMVR delivery procedure then proceeds in accordance with the instructions for use of the TENDYNE TMVR by dilating the access site and introducing a balloon catheter over the guidewire to the left ventricle and left atrium 522, advancing the guidewire through the mitral valve annulus and into the left atrium 524, deflating and removing the balloon catheter leaving the guidewire positioned in the left atrium 526, delivering the TMVR delivery system over the guidewire and into the left atrium 528, positioning the TMVR above the mitral valve annulus 530, collapsing the delivery system dilator and removing the guidewire 532, deploying the TMVR in the mitral valve annulus 534, confirming positioning and repositioning the TMVR if needed while maintaining tension on the TMVR tether bridle 330 connected to the tethers 536, advancing an apical pad and locking anchor over the tether bridle 330 538, adjusting tether tension and verifying TMVR performance and position 540, locking the apical pad to the access port with the locking anchor 542, and then trimming the tether bridle 330 to a desired length.

Finally, sensors and/or power sources 44 may, optionally, be added to the access assembly 45 to sense physiologically conditions in the body, such as within the heart, on the surface of the heart, act as on-board pacemaker or as signal inputs to a VAD. Other sensor and/or powered devices that may be employed with the access assembly include, without limitation, neurostimulators, cochlear implants, implantable cardiac defibrillators, cardiac resynchronization devices, drug delivery systems, and bone growth generators. Currently, power sources for implantable medical devices tend to be rechargeable batteries, typically lithium-ion batteries, but other power sources such, for example, as thermoelectric devices that rely upon temperature gradients to supply power are also contemplated and intended in the present disclosure. The sensors and/or power sources 44 may be configured to be coupled to or embedded in any or all of the following components of the access assembly: the attachment skirt 24, the first annular cap member 31, the second annular cap member 32, the annular ring 12 of the access cuff 10, and/or the tissue anchors. Further, the sensors and/or power sources 44 may be embedded in a dielectric material, such as PTFE or a polyimide, commonly known as KAPTON, and formed into an annular ring that may be positioned between the anatomic tissue surface and the attachment skirt 24. A wide variety of sensors 44 may be employed to sense a wide variety of physiological conditions, including without limitation, temperature, pressure, electrical signals, nerve signals, physiological chemistries, flow rates, or the like.

As noted above, the disclosed variants of access assembly 45 may also be used in a wide variety of non-vascular medical applications to create end-to-side conduits between anatomic passageways or between a tubular conduit and an anatomic passageway. Those skilled in the art will appreciate and understand that the scope of utility and the scope of the constructs of the end-to-side assemblies of the present disclosure described herein may have a large number of variations and that the scope of the invention is limited only by the claims appended hereto.

1. An access port, comprising an annular ring, a central opening passing through the annular ring, an optional tubular projection extending from the annular ring and coaxial with the central opening, an annular skirt on a distal surface of the annular ring and co-axial with the central opening; a plurality of tissue openings passing axially through the annular ring and spaced apart about a circumference of the annular ring; and a plurality of tissue anchors configured to axially pass into and through the plurality of tissue openings, the annular skirt, and into anatomic tissue to exert an axially compressive force to the annular ring and the access skirt against the anatomic tissue.

2. The access port of claim 1, further comprising an annular access cap having a central opening co-axial with the central opening of the annular ring.

3. The access port of claim 2, wherein the annular access cap further comprises a first annular cap member that removably engages with the annular ring of the access port, a second annular cap member coupled to the first annular cap member, wherein the first annular access cap member and the second annular access cap member each have a central opening defining the central opening of the annular access cap.

4. The access port of claim 3, wherein further comprising a seal disposed within the central opening.

5. The access port of claim 1, wherein the tissue anchors are selected from the group of tissue screws, expandable pins, and expandable anchors.

6. The access port of claim 2, wherein the annular access cap further comprises a tubular projection disposed in the central opening of the annular access cap.

7. The access port of claim 6, wherein the tubular projection is configured to project distally from the central opening of the annular access cap and pass into and through a penetration into the anatomic tissue.

8. The access port of claim 2, wherein the annular access cap further comprises a sealing plug projecting distally from the central opening of the annular access cap.

9. The access port of claim 8, wherein the sealing plug has a length less than or equal to a thickness of the anatomic tissue.

10. The access port of claim 3, wherein the annular access cap further comprises a valve.

11. The access port of claim 10, wherein the valve is a one-way valve configured to pass medical instruments and/or medical devices therethrough.

12. The access port of claim 11, further comprising a sealing plug passing through the one-way valve, the sealing plug being retained by the one-way valve and the second annular cap member.

13. The access port of claim 1, further comprising an access port delivery system comprising a drive input sub-system, a synchronous drive gear subsystem, a driver sub-system, and a central bore passing along an entire longitudinal axis of the access port delivery system and open at opposing ends thereof, wherein the access port is removably coupled to the driver sub-system.

14. The access port delivery system of claim 13, wherein the tissue anchors are removably coupled to the driver sub-system.

15. The access port delivery system of claim 13, further comprising a rotational member coupled to the drive input sub-system and the central bore passes through the rotational member and is accessible there through.

16. The access port of claim 2, further comprising at least one sensor and/or power source.

17. A method for delivering a transcatheter valve prosthesis to a valve annulus within a heart, comprising the steps of:
establishing a target on the ventricular apex of the heart muscle;
advancing an access port delivery tool carrying an access port and contacting the surface of the ventricular apex;
affixing the access port to the ventricular apex by synchronously driving a plurality of tissue anchors into and through the access port;
defining an access site opening within a central opening of the access port;
inserting a valve prosthesis delivery system through the access site opening;
deploying and positioning the valve prosthesis within the valve annulus;
retaining the position of the valve prosthesis while withdrawing the valve prosthesis delivery system; and
anchoring the valve prosthesis with respect to the access port and sealing the central opening of the access port.

18. The method of claim 17, wherein the step inserting a valve prosthesis further comprises the step of delivering the valve prosthesis through a central bore of the access port delivery tool while the access port delivery tool is coupled to the access port.

19. The method of claim 17, further comprising the step of placing a guidewire at the established target on the ventricular apex of the heart muscle.

20. The method of claim 17, further comprising the step of coring heart tissue within the central opening of the access port.
